(19) United States
(12) Patent Application Publication
(10) Pub. No.: US 2025/0085829 A1
(43) Pub. Date: Mar. 13, 2025

(54) MEDICAL INFORMATION PROCESSING SYSTEM, MEDICAL INFORMATION PROCESSING METHOD, AND PROGRAM

(71) Applicant: SONY GROUP CORPORATION, TOKYO (JP)

(72) Inventors: KANA MATSUURA, TOKYO (JP); TAKUYA NAKAMURA, TOKYO (JP); TAKESHI MAEDA, TOKYO (JP)

(21) Appl. No.: 18/292,435

(22) PCT Filed: Mar. 8, 2022
(86) PCT No.: PCT/JP2022/009957

§ 371 Date: Jan. 26, 2024

(30) Foreign Application Priority Data
Aug. 27, 2021 (JP) 2021-138530

(51) Int. Cl. G06F 3/04817; G16H 40/20

(52) U.S. Cl. CPC G06F 3/04817; G16H 40/20

(57) ABSTRACT

The present disclosure relates to a medical information processing system, a medical information processing method, and a program for enabling visualization of situations of entire operating rooms and situations of the respective medical staff members. A medical information processing system is provided, and the medical information processing system includes: a calculation unit that calculates the amount of activity in entire operating rooms and the amount of activity of each medical staff member, on the basis of operating room information including operating room camera images; and a generation unit that generates operating room graphics including graphics indicating a temporal change in the amount of activity in the entire operating rooms, and medical staff graphics including graphics indicating each medical staff member, the medical staff graphics visually changing with the amount of activity. The present disclosure can be applied to a system that manages the operating rooms in a hospital, for example.

### TECHNICAL FIELD

The present disclosure relates to a medical information processing system, a medical information processing method, and a program, and more particularly, to a medical information processing system, a medical information processing method, and a program for enabling visualization of situations of entire operating rooms and situations of the respective medical staff members.

### BACKGROUND ART

In an operating room, it is difficult to accurately estimate the amount of activity required in one medical operation, because of a patient's condition, a device failure, or the like. Therefore, in a case where manpower is required more than expected due to difficulty in a medical operation or the like, the medical staff in each operating room requests for help from the administrative staff in a central medical center, and the administrative staff in the central medical center reviews the medical staff assignment by checking the state of each operating room. A system useful for reviewing such medical staff assignment has been suggested (see Patent Document 1, for example).

### CITATION LIST

### Patent Document

Patent Document 1: Japanese Patent Application Laid-Open No. 2009-230535

### SUMMARY OF THE INVENTION

### Problems to be Solved by the Invention

When the amount of activity in entire operating rooms changes due to factors such as a patient's condition, the amount of activity of each medical staff member also changes. On the other hand, a change in the amount of activity in the entire operating rooms is not necessarily apparent only from the amounts of activity of the respective medical staff members. For example, there might be a case where a medical staff member is performing, in a hurry, a task that does not greatly affect the progress of a medical operation, though the entire operating rooms are calm. Also, there might be a case where the amount of activity in the entire operating rooms is large, but the amount of activity in the task of each medical staff member is small. Therefore, it is required to visualize situations of the entire operating rooms and situations of the respective medical staff members.

The present disclosure has been made in view of such circumstances, and is to enable visualization of situations of the entire operating rooms and situations of the respective medical staff members.

### Solutions to Problems

A medical information processing system according to one aspect of the present disclosure is a medical information processing system that includes: a calculation unit that calculates the amount of activity in entire operating rooms and the amount of activity of each medical staff member, on the basis of operating room information including operating room camera images; and a generation unit that generates operating room graphics including graphics indicating a temporal change in the amount of activity in the entire operating rooms, and medical staff graphics including graphics indicating each medical staff member, the medical staff graphics visually changing with the amount of activity.

A medical information processing method according to one aspect of the present disclosure is a medical information processing method that is implemented in a medical information processing system, and includes: calculating the amount of activity in entire operating rooms and the amount of activity of each medical staff member, on the basis of operating room information including operating room camera images; and generating operating room graphics including graphics indicating a temporal change in the amount of activity in the entire operating rooms, and medical staff graphics including graphics indicating each medical staff member, the medical staff graphics visually changing with the amount of activity.

A program according to one aspect of the present disclosure is a program for causing a computer to function as: a calculation unit that calculates the amount of activity in entire operating rooms and the amount of activity of each medical staff member, on the basis of operating room information including operating room camera images; and a generation unit that generates operating room graphics including graphics indicating a temporal change in the amount of activity in the entire operating rooms, and medical staff graphics including graphics indicating each medical staff member, the medical staff graphics visually changing with the amount of activity.

In the medical information processing system, the medical information processing method, and the program according to one aspect of the present disclosure, the amount of activity in the entire operating rooms and the amount of activity of each medical staff member are calculated on the basis of operating room information including operating room camera images; and operating room graphics including graphics indicating a temporal change in the amount of activity in the entire operating rooms, and medical staff graphics including graphics indicating each medical staff member are generated, the medical staff graphics visually changing with the amount of activity.

Note that the medical information processing system according to one aspect of the present disclosure may be a system including a plurality of devices, may be an independent device, or may be an internal block in one device.

### BRIEF DESCRIPTION OF DRAWINGS

FIG. 1 is a block diagram illustrating an example configuration of an embodiment of a medical information processing system to which the present disclosure is applied.

FIG. 2 is a block diagram illustrating an example configuration of an information processing device illustrated in FIG. 1.

FIG. 3 is a diagram illustrating an example structure of a screen displayed on the display device illustrated in FIG. 1.

FIG. 4 is a diagram illustrating an example of display of an operation floor screen.

FIG. 5 is a diagram illustrating details of area information.

FIG. 6 is a diagram illustrating another example of display of an operation floor screen.

FIG. 7 is a diagram illustrating another example of display of an operation floor screen.

FIG. 8 is a diagram illustrating an example of display of an area detail screen.

FIG. 9 is a diagram illustrating an example of handling of an area detail screen.

FIG. 10 is a diagram illustrating another example of display of an area detail screen.

FIG. 11 is a diagram illustrating an example of display of a staff detail screen.

FIG. 12 is a diagram illustrating another example of display of a staff detail screen.

FIG. 13 is a diagram illustrating an example of one-screen display of information.

FIG. 14 is a diagram illustrating an example of display of medical staff icons.

FIG. 15 is a flowchart for explaining a flow in a process to be performed by the medical information processing system.

FIG. 16 is a flowchart for explaining a detailed flow in a medical staff activity amount calculation process.

FIG. 17 is a flowchart for explaining a detailed flow in an operating room total activity amount calculation process.

FIG. 18 is a block diagram illustrating an example configuration of a computer.

### MODE FOR CARRYING OUT THE INVENTION

### 1. Embodiment of the Present Disclosure

### (Outline of a System)

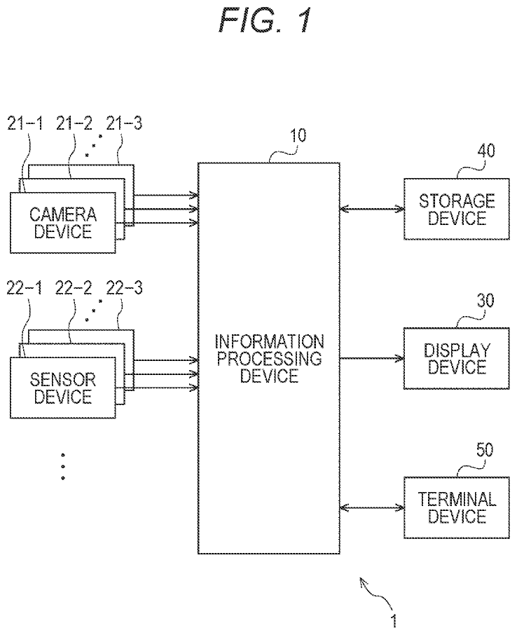

FIG. 1 is a block diagram illustrating an example configuration of an embodiment of a medical information processing system to which the present disclosure is applied.

In FIG. 1, a medical information processing system 1 includes an information processing device 10, camera devices 21-1 to 21-M (M: an integer of 1 or greater), sensor devices 22-1 to 22-N(N: an integer of 1 or greater), a display device 30, a storage device 40, and a terminal device 50.

The medical information processing system 1 is a system constructed in a hospital, and the information processing device 10 is mutually connected to each of the camera devices 21-1 to 21-M, the sensor devices 22-1 to 22-N, the display device 30, the storage device 40, and the terminal device 50 via a network such as an in-hospital local area network (LAN).

The camera devices 21-1 to 21-M are cameras capable of capturing moving images or still images. Each of the camera devices 21-1 to 21-M captures an image of the inside of an operating room, and outputs camera image data thus obtained to the information processing device 10.

The camera devices 21-1 to 21-M are installed in an operating room in the hospital. At least one camera device 21 is installed in each operating room in the hospital. Each of the camera devices 21-1 to 21-M may be installed in an operating room in the hospital, or may be included in a device or the like worn by a medical staff member.

The sensor devices 22-1 to 22-N include various sensors such as an acceleration sensor and a distance sensor. Each of the sensor devices 22-1 to 22-N performs sensing, and outputs sensing data obtained by the sensing to the information processing device 10.

Each of the sensor devices 22-1 to 22-N is used as appropriate. Each of the sensor devices 22-1 to 22-N may be installed in an operating room in the hospital, or may be included in a device worn by a medical staff member. For example, an acceleration sensor or radio frequency identification (RFID) is provided as a physical attachment, so that the position and movement of a person or an object can be sensed.

The camera image data and the sensing data are output as operating room information to the information processing device 10 via the network. The operating room information is information regarding the operating room in the hospital. The camera image data includes data of an operating room bird's-eye view video image captured by a plurality of the camera devices 21, data of a subjective camera image obtained by imaging an individual's hand, and the like. The sensing data includes acceleration data measured by an acceleration sensor, distance measurement data measured by a distance sensor, and the like.

Note that, in FIG. 1, camera image data and sensing data are illustrated as an example of the operating room information, but data obtained from other devices may be used as the operating room information. The other devices include medical devices installed in the operating room, the storage device 40, the terminal device 50, and the like.

The information processing device 10 is a device such as a server or a personal computer (PC). The information processing device 10 is installed in a room such as a server room in the hospital or a central medical administration room for managing the operating rooms in the hospital.

The information processing device 10 receives, as the operating room information, the camera image data from the camera devices 21-1 to 21-M, the sensing data from the sensor devices 22-1 to 22-N, and the like. On the basis of the operating room information, the information processing device 10 generates graphics corresponding to the amount of activity in the entire operating rooms and the amount of activity of each medical staff member, and outputs the graphics to the display device 30.

The display device 30 is a device such as a PC or a display device. The display device 30 is installed in a room such as the central medical administration room in the hospital. The display device 30 displays a screen including the graphics output from the information processing device 10.

The storage device 40 is a device such as a server or a storage. The storage device 40 is installed in a room such as a server room in the hospital. The storage device 40 records a hospital database, and provides data stored in the hospital database in response to a request from the information processing device 10.

The terminal device 50 is a device such as a mobile terminal like a personal handy-phone system (PHS), or a wearable terminal like a head mounted display (HMD) or an eyeglasses-type device. The terminal device 50 is worn by a medical staff member in the operating room. The terminal device 50 performs processing according to the data output from the information processing device 10.

In the medical information processing system 1 designed as described above, the information processing device 10 performs processing based on the operating room information from the camera devices 21-1 to 21-M, the sensor devices 22-1 to 22-N, and the like, and a screen including the graphics corresponding to the result of the processing is displayed on the display device 30.

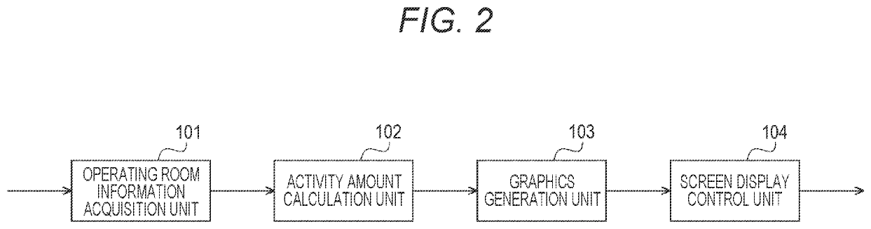

FIG. 2 is a block diagram illustrating an example functional configuration of the information processing device 10 illustrated in FIG. 1.

In FIG. 2, the information processing device 10 includes an operating room information acquisition unit 101, an activity amount calculation unit 102, a graphics generation unit 103, and a screen display control unit 104.

The operating room information acquisition unit 101 acquires operating room information, and supplies the operating room information to the activity amount calculation unit 102. For example, the operating room information can include sensing data from the sensor devices 22-1 to 22-N, and the like, in addition to camera image data from the camera devices 21-1 to 21-M.

On the basis of the operating room information supplied from the operating room information acquisition unit 101, the activity amount calculation unit 102 calculates the amount of activity in the entire operating rooms in the hospital and the amount of activity of each medical staff member in each operating room, and supplies the activity amounts to the graphics generation unit 103.

The amount of activity in the entire operating rooms is calculated from an index value indicating busyness in each operating room in the hospital, a predicted value of busyness in each operating room, and the like. The amount of activity of each medical staff member is calculated from an index value indicating busyness of each medical staff member in each operating room, a predicted value of busyness of each medical staff member, and the like.

On the basis of the activity amounts supplied from the activity amount calculation unit 102, the graphics generation unit 103 generates operating room graphics and medical staff graphics, and supplies the graphics to the screen display control unit 104.

The operating room graphics are graphics based on the amount of activity in the entire operating rooms, and include graphics indicating a temporal change in the amount of activity in the entire operating rooms. The medical staff graphics are graphics based on the amount of activity of the medical staff, and include graphics indicating each member of the medical staff that visually changes depending on the amount of activity.

The screen display control unit 104 displays, on the display device 30, a screen including the graphics supplied from the graphics generation unit 103. The screen to be displayed on the display device 30 includes at least either the operating room graphics or the medical staff graphics.

### (Screen Structure)

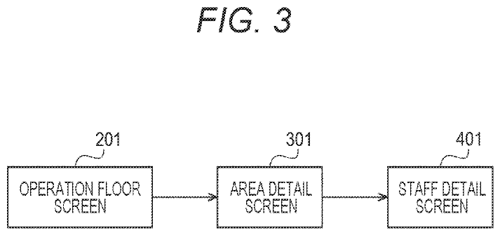

FIG. 3 is a diagram illustrating an example structure of a screen displayed on the display device 30 illustrated in FIG. 1.

An administrative staff member at the central medical administration room monitors the state of each operating room through the screen displayed on the display device 30. An administrative staff member can handle the screen displayed on the display device 30, using an input device such as a mouse. The screens to be displayed on the display device 30 include an operation floor screen 201, an area detail screen 301, and a staff detail screen 401.

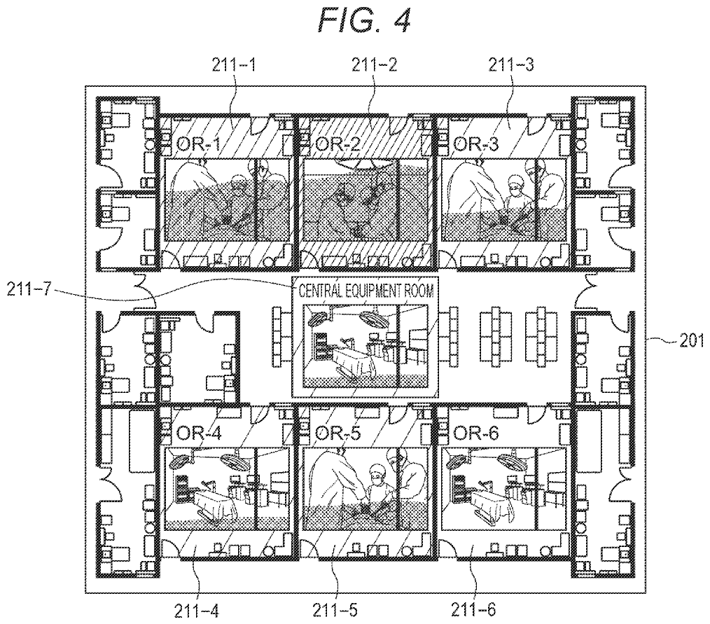

The operation floor screen 201 includes graphics indicating a temporal change in the amount of activity in the entire operating rooms in the hospital. The operation floor screen 201 displays graphics indicating a temporal change in the amount of activity in each operating room in each of the areas corresponding to the operating rooms. The operation floor screen 201 will be described later in detail, with reference to FIGS. 4 to 7.

In a case where an area is selected by an administrative staff member clicking the position of the desired area on the operation floor screen 201, the area detail screen 301 related to the selected area is displayed.

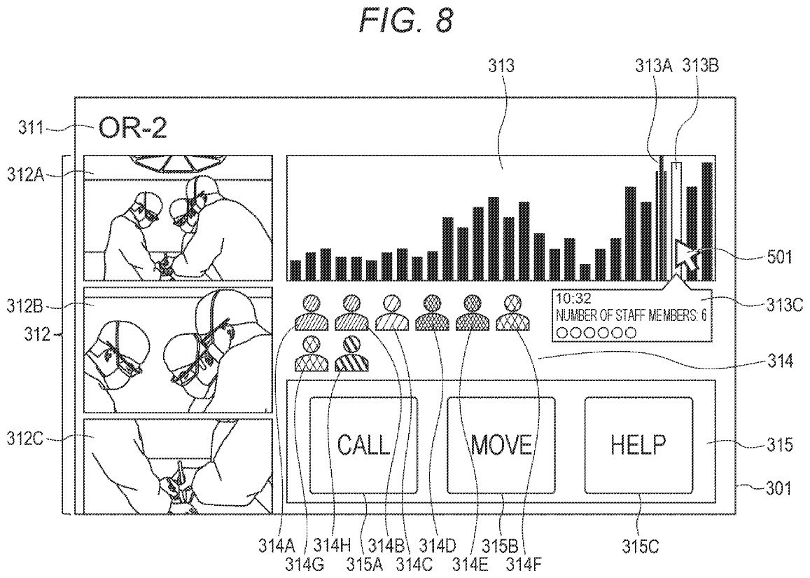

The area detail screen 301 includes graphics indicating a temporal change in the amount of activity in an operating room (the target operating room) in the selected area, and graphics indicating the amount of activity of each medical staff member in the target operating room. The area detail screen 301 displays medical staff icons representing the respective medical staff members. The area detail screen 301 will be described later in detail, with reference to FIGS. 8 to 10.

In a case where a medical staff member is selected by an administrative staff member clicking the desired medical staff icon on the area detail screen 301, the staff detail screen 401 related to the selected medical staff member is displayed.

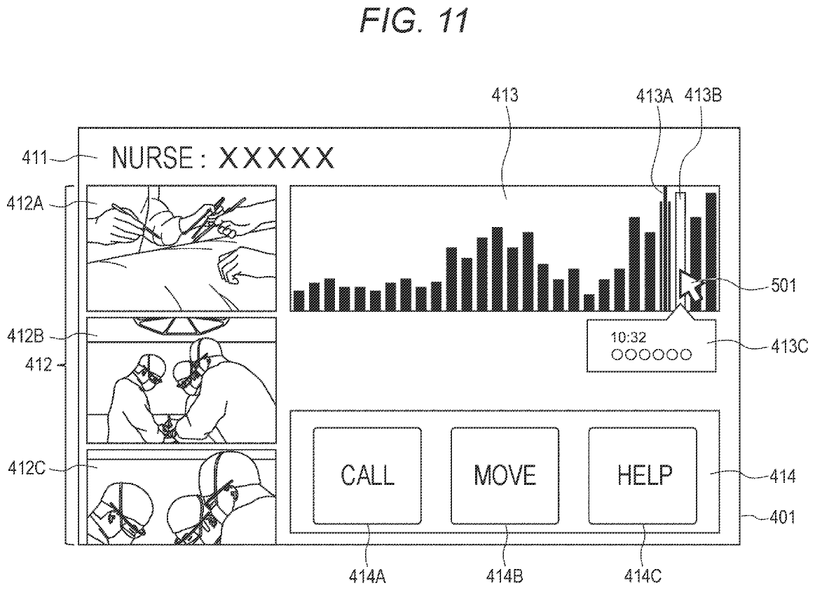

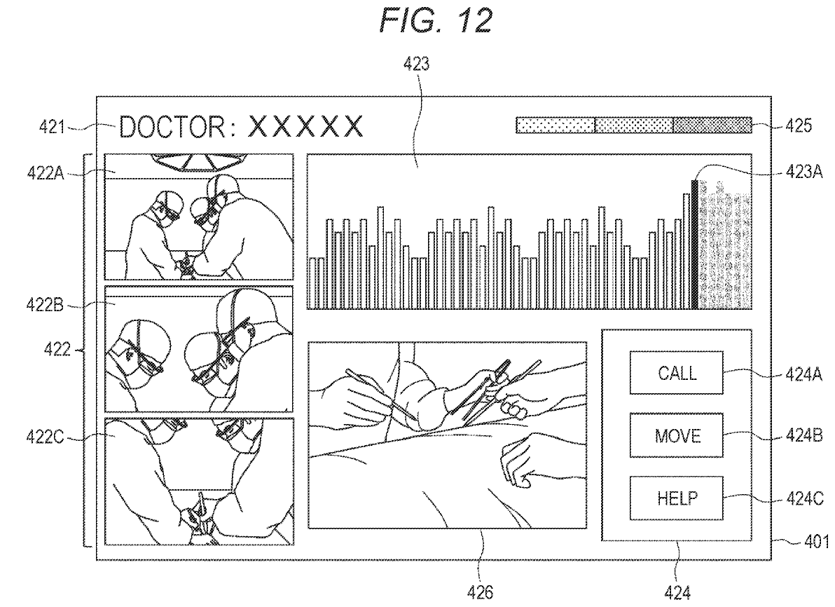

The staff detail screen 401 includes graphics indicating the amount of activity of the selected medical staff member (the target medical staff member). The staff detail screen 401 will be described later in detail, with reference to FIGS. 11 and 12.

Note that, in a case where the operation floor screen 201 is in a mode for simultaneously displaying a plurality of operating rooms, when an operating room is selected by an administrative staff member, the area detail screen 301 related to the operating room may be displayed, and, when a medical staff member is selected by an administrative staff member, the staff detail screen 401 related to the medical staff member may be displayed.

### (Operation Floor Screen)

FIG. 4 is a diagram illustrating an example of display of the operation floor screen 201.

In FIG. 4, the operation floor screen 201 includes graphics indicating a temporal change in the amount of activity in the entire operating rooms in the hospital.

In a case where there are six operating rooms OR-1 to OR-6 in a hospital floor plan, area information 211-1 is superimposed on the area corresponding to the operating room OR-1 on the operation floor screen 201. The area information 211-1 includes a camera image obtained by imaging the inside of the operating room OR-1 and a graph superimposed on the camera image. This graph indicates a temporal change in the amount of activity in the operating room OR-1, and indicates the busyness in the past, the present, and the future in the operating room OR-1.

Likewise, the respective sets of area information 211-2 to area information 211-6 are superimposed on the areas corresponding to the operating rooms OR-2 to OR-6. In the area information 211-2 to 211-6, a graph indicating a temporal change in the amount of activity in the operating room is superimposed on a camera image obtained by imaging the operating room corresponding to each area, as in the area information 211-1.

In the area information 211-1 to 211-6, the background in the camera image of each operating room is a background color corresponding to the amount of activity in the operating room. For example, as for the stage corresponding to the amount of the current or future activity in each operating room, the background color can be expressed in the four stages of red (a reddish color), orange (an orangish color), yellow (a yellowish color), and green (a greenish color), the busiest stage being set to red (a reddish color), the least busy stage is set to green (a greenish color). These are techniques that are used in so-called contour charts.

More specifically, in a case where it is determined that the situation is the busiest on the basis of the amount of activity in the operating room OR-2, the background color in the area information 211-2 changes to red. In the busiest situation such as the above, a display element (a background, a graph, or the like) in the area information 211-2 may be displayed as a more conspicuous alert that is blinking or the like.

Meanwhile, in a case where it is determined that the situation is quite busy on the basis of the amount of activity in the operating room OR-1, the background color in the area information 211-1 changes to orange. In a case where it is determined that the situation is not very busy on the basis of the amounts of activity in the operating room OR-3 and the operating room OR-5, the background colors in the area information 211-3 and the area information 211-5 change to yellow.

Further, in a case where it is determined that the situation is unbusy on the basis of the amount of activity in the operating room OR-4, the background color in the area information 211-4 changes to green. Note that, since the operating room OR-6 is an operating room that is not currently used, the background color in the area information 211-6 can be any predetermined color such as gray.

On the operation floor screen 201 in FIG. 4, the differences in background color among the respective sets of the area information 211 are represented by hatched patterns. As the determination process for representing the background colors in four stages, threshold determination by comparing the amount of activity in each operating room with a threshold may be performed, and the situation of each operating room may be classified as one of the four stages, for example. Alternatively, the amount of activity in each operating room may be compared with the amounts of activity in the other operating rooms, and the situation of each operating room may be classified as one of the four stages.

Note that the stages of the background colors are not limited to four stages, and may be classified in into any plurality of stages, such as three or fewer stages, or five or more stages. In that case, the background colors corresponding to the classified stages are used. Also, the background color can be determined from at least one of the amounts of the past, present, and future activity in each operating room. Further, as long as the busyness in each operating room can be visually recognized, the busyness may be expressed in any display form other than background color.

As described above, on the operation floor screen 201, the graph superimposed on the camera image of each operating room arranged in the area information 211-1 to 211-6 indicates the temporal change in the amount of activity in the entire operating rooms in the hospital, and the background in the camera image of each operating room is shown in the background color corresponding to the situation of each operating room. Thus, the administrative staff can visually recognize the busyness in each operating room by checking the operation floor screen 201.

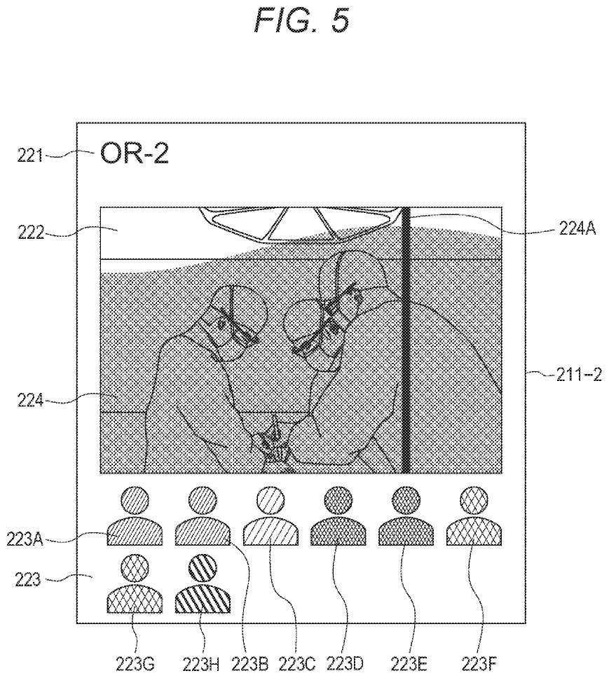

Here, the area information 211 is described in detail with reference to FIG. 5. FIG. 5 illustrates the area information 211-2 in FIG. 4 as an example. Since the area information 211-2 is determined to be in the busiest situation from the amount of activity in the operating room OR-2, the background color is red (a reddish color). The area information 211-2 includes an area name 221, a camera image 222, medical staff icons 223, and an activity status graph 224.

In the area name 221, “OR-2”, which is the name of the area corresponding to the operating room OR-2 is displayed. As the area name 221, information such as an operative method indicating a surgical operation procedure may be displayed together with the name of the area.

A representative camera image among the camera images captured in the operating room OR-2 is displayed as the camera image 222. The activity status graph 224 indicating a temporal change in the amount of activity in the operating room OR-2 is superimposed on the camera image 222. The activity status graph 224 is a graph in which the abscissa axis indicates time, and the ordinate axis indicates the amount of activity, and is represented by a pattern superimposed on the camera image 222. In the activity status graph 224, the current time is represented by a vertical bar 224A. With the reference amount of activity being the amount of the current activity indicated by the vertical bar 224A, the left side indicates the amount of the past activity, and the right side indicates the amount (a predicted value) of the future activity.

The medical staff icons 223 are icons representing the respective medical staff members such as the doctors and the nurses in the operating room OR-2. As there are eight medical staff members in the operating room OR-2 at the present moment, medical staff icons 223A to 223H corresponding to the respective medical staff members are displayed. The medical staff icons 223A to 223H are colored for the respective roles of the doctors, the nurses, and the like.

Note that, in addition to the colors for the respective roles, characters, symbols, and the like indicating doctors, nurses, and the like may be added to express the respective roles in other display forms. Alternatively, when a predetermined action such as a click action is performed on a medical staff icon 223, the name of the corresponding medical staff member in the operating room OR-2 may be displayed.

Although the area information 211-2 has been described as a representative example herein, an area name 221, a camera image 222, medical staff icons 223, and an activity status graph 224 are also displayed for each set of the area information 211-1 and 211-3 to 211-6 in FIG. 4 in accordance with the respective operating rooms.

Note that, on the operation floor screen 201, the area information 211 may be superimposed not only on the operating rooms but also on a room such as a central equipment room for cleaning, drying, sterilizing, storing, and the like of medical equipment used in the operating rooms. In FIG. 4, area information 211-7 is superimposed on the area corresponding to the central equipment room.

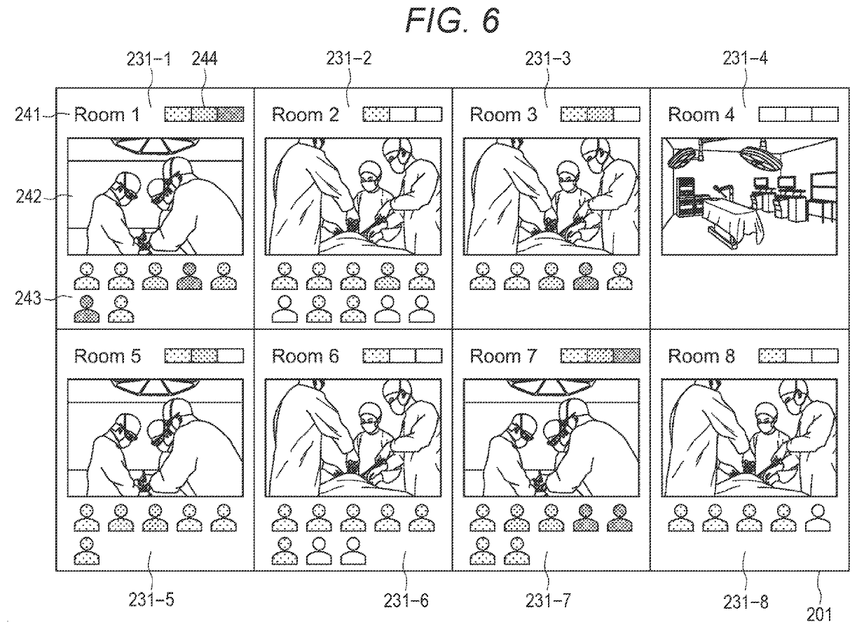

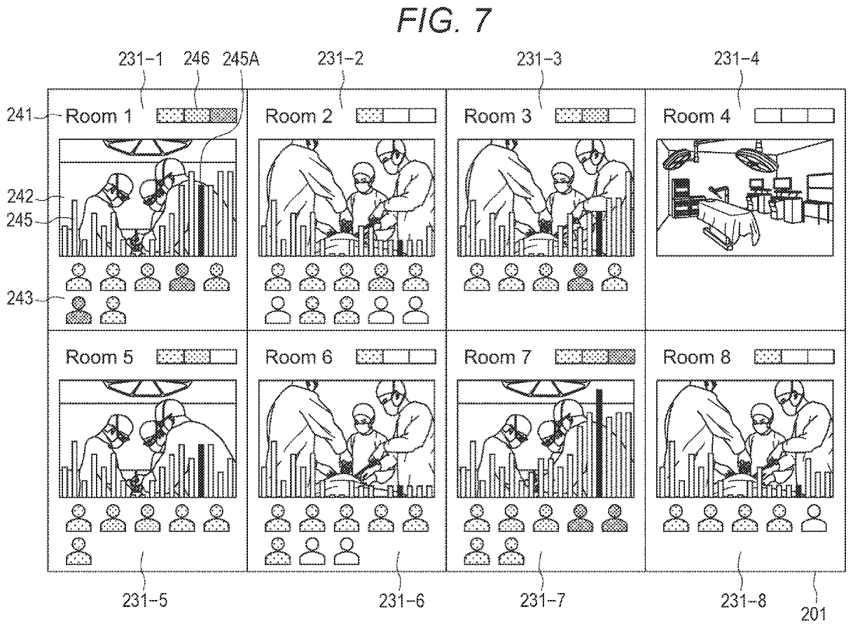

The display form of the operation floor screen 201 illustrated in FIG. 4 is an example, and some other display form may be adopted. FIGS. 6 and 7 illustrate another example of display of the operation floor screen 201.

In FIG. 6, in a case where there are eight operating rooms in the hospital, area information 231-1 to 231-8 corresponding to the respective operating rooms is displayed on the operation floor screen 201. For example, the area information 231-1 includes an area name 241, a camera image 242, medical staff icons 243, and a status gauge 244.

In the area name 241, “Room 1”, which is the name of the area corresponding to an operating room #1 is displayed. A representative camera image among the camera images captured in the operating room #1 is displayed as the camera image 242.

The medical staff icons 243 are icons representing the respective medical staff members such as the doctors and the nurses in the operating room #1. As there are seven medical staff members in the operating room #1 at the present moment, seven human-shaped icons (shaped like human upper bodies) are displayed. The medical staff icons 243 are colored in accordance with the amounts of activity of the corresponding medical staff members.

For example, the human-shaped icon corresponding to a medical staff member in a busy state is in red (a reddish color), and the human-shaped icon corresponding to a medical staff member in an unbusy state is in green (a greenish color). As the number of medical staff members in the operating room #1, and the busyness of each medical staff members are indicated by the medical staff icons 243, the administrative staff can visually recognize the busyness of each medical staff member in each operating room. Note that, as long as the busyness of each medical staff member can be visually recognized, the busyness may be expressed in any display form other than color.

The status gauge 244 indicates the amount of activity in the operating room #1 in three stages. For example, the area information 231-1 indicates that the status gauge 244 has risen to the highest of the three stages, and the operating room is in a busy situation at the present moment or in the future.

Although the area information 231-1 has been described herein as a representative example on the operation floor screen 201 in FIG. 6, an area name 241, a camera image 242, medical staff icons 243, and a status gauge 244 are also displayed for each set of the area information 231-2 to 231-8 in accordance with the respective operating rooms.

As described above, on the operation floor screen 201 in FIG. 6, the area information 231-1 to 231-8 indicate a temporal change in the amount of activity in the entire operating rooms in the hospital, so that the administrative staff can visually recognize the busyness in each operating room. For example, the operation floor screen 201 in FIG. 6 shows that the operating room #1 (Room 1) and an operating room #7 (Room 7) are in the busiest situation, and many medical staff members are in a busy state. The operation floor screen 201 also shows that an operating room #3 (Room 3) and an operating room #5 (Room 5) are in a slightly busy situation, and the medical staff is also slightly busy there, while an operating room #2 (Room 2), an operating room #6 (Room 6), and an operating room #8 (Room 8) are in a relatively unbusy situation, and the medical staff is also unbusy.

The operation floor screen 201 in FIG. 7 differs from the operation floor screen 201 in FIG. 6 in that an activity status graph 245 indicating a temporal change in the amount of activity in the operating room #1 is superimposed on the camera image 242 in the area information 231-1. The activity status graph 245 is a graph in which the abscissa axis indicates time, and the ordinate axis indicates the amount of activity in the operating room #1, and is represented by a bar chart superimposed on the camera image 242. In the activity status graph 245, a bar 245A shown in a color different from the color of the other bars in the bar chart indicates the amount of activity at the current time. The left side of the bar 245A indicates the amount of the past activity, and the right side indicates the amount (a predicted value) of the future activity.

Note that, in the activity status graph 245, a vertical bar indicating the current time may also be displayed. In a case where an administrative staff member performs an action to move the vertical bar representing the current time to the right side, the colors of the medical staff icons 243 may be changed on the basis of the amounts of the future activity of the respective medical staff members in accordance with the time indicated by the vertical bar.

Further, in the area information 231-1 on the operation floor screen 201, a status gauge 246 that indicates the progress status of the operating room in three stages is displayed. For example, the area information 231-1 indicates that the status gauge 246 has risen to the highest of the three stages, and the medical operation is close to the end.

For example, the progress status of an operating room is obtained as follows. Data regarding medical operation contents stored in the hospital database in the storage device 40, and which stage of the medical operation flow the current operation status is in (for example, how many percentages of the entire medical operation has been completed) is calculated from a result of analysis of the camera image and the like in the medical operation flow associated with the acquired medical operation contents.

Although the area information 231-1 has been described herein as a representative example on the operation floor screen 201 in FIG. 7, an area name 241, a camera image 242, medical staff icons 243, an activity status graph 245, and a status gauge 246 are also displayed for each set of the area information 231-2 to 231-8 in accordance with the respective operating rooms.

Note that, although the status gauge 244 indicates the amount of activity in an operating room in a stepwise manner on the operation floor screen 201 in FIG. 6, the status gauge 244 may indicate the progress status of an operating room in a stepwise manner like the status gauge 246. For example, the status gauge 244 can indicate the progress status of an operating room in a stepwise manner, instead of the amount of activity in the operating room or together with the amount of activity in the operating room. The number of stages in the status gauge 244 and the status gauge 246 is not limited to three stages, and may be expressed as some other number of stages, such as two stages, or four or more stages. Further, as long as the amounts of activity in the operating rooms and the progress statuses of the operating rooms can be visually recognized, a display form other than a gauge may be adopted.

Furthermore, the layout of the operation floor screen 201 can be changed. For example, the number of operating rooms varies with each hospital, and therefore, display of the operation floor screen 201 can be switched in accordance with the parameters of the operating rooms or the like.

As described above, on the operation floor screen 201, the camera image 222 (242) of each operating room and the medical staff icons 223 (243) representing the respective medical staff members in the operating room are arranged for each operating room, and the activity status graph 224 (245) indicating a temporal change in the amount of activity in the operating room is superimposed on the camera image 222 (242) of the operating room. Also, on the operation floor screen 201, the background in the camera image 222 (242) of each operating room is set in a display form (a color or the like) corresponding to the amount of activity in the operating room. The medical staff icons 243 visually change with the amounts of activity of the respective medical staff members. Further, the medical staff icons 223 are in different display forms (colors or the like) in accordance with the roles of the respective medical staff members.

### (Area Detail Screen)

FIG. 8 is a diagram illustrating an example of display of the area detail screen 301.

In FIG. 8, the area detail screen 301 includes graphics indicating a temporal change in the amount of activity in the target operating room, and graphics indicating the amount of activity of each medical staff member in the operating room.

The area detail screen 301 in FIG. 8 is a screen that is displayed in a case where predetermined actions such as selecting the area corresponding to the operating room OR-2 on the operation floor screen 201 in FIG. 4 and performing a click action are performed. Since the area detail screen 301 in FIG. 8 is determined to be in the busiest situation from the amount of activity in the operating room OR-2, the background color is red (a reddish color).

The area detail screen 301 includes an area name 311, a camera image group 312, an activity status graph 313, medical staff icons 314, and an instruction button group 315.

In the area name 311, “OR-2”, which is the name of the area corresponding to the operating room OR-2 is displayed. As the area name 311, information such as an operative method may be displayed together with the name of the area.

A representative camera image 312A among the camera images captured in the operating room OR-2 is disposed in the camera image group 312. Camera images 312B and 312C at different angles from the camera image 312A are also disposed in the camera image group 312. The camera images arranged in the camera image group 312 may include an image captured by an endoscope camera or the like, in addition to images obtained by imaging the situation in the operating room OR-2.

The activity status graph 313 is a graph indicating the amount of activity in the operating room OR-2 in chronological order, the abscissa axis indicating time, the ordinate axis indicating the amount of activity. In the activity status graph 313, the current time is represented by a vertical bar 313A. With the reference amount of activity being the amount of the current activity indicated by the vertical bar 313A, the left side indicates the amount of the past activity, and the right side indicates the amount of the future activity.

In a case where a pointer 501 is operated by an administrative staff member, and a desired bar 313B in the activity status graph 313 is selected, detailed information 313C regarding the operating room OR-2 indicated by the bar 313B is displayed. The detailed information 313C includes time information, information regarding the number of medical staff members, and the like. In this manner, in addition to the amount of activity in the operating room, numerical information indicating the situation of the operating room can be displayed in a so-called dashboard form.

The medical staff icons 314 are icons representing the respective medical staff members such as the doctors and the nurses in the operating room OR-2. As there are eight medical staff members in the operating room OR-2 at the present moment, medical staff icons 314A to 314H corresponding to the respective medical staff members are displayed. The medical staff icons 314A to 314H are colored for the respective roles of the doctors, the nurses, and the like.

Note that, in addition to the colors for the respective roles, characters, symbols, and the like indicating doctors, nurses, and the like may be added to express the respective roles in other display forms. Alternatively, when a predetermined action such as a click action is performed on a medical staff icon 314, the name of the corresponding medical staff member in the operating room OR-2 may be displayed.

In the instruction button group 315, a button group to be handled when the administrative staff gives an instruction to the medical staff in each operating room is disposed. The instruction button group 315 includes a call button 315A, a move button 315B, and a help button 315C.

The call button 315A is a button to be handled when a call to a medical staff member as the call target is made. For example, an administrative staff member selects a medical staff member displayed in the camera images arranged in the camera image group 312 by a pointer action or the like, and presses the call button 315A, to be connected to the terminal device 50 worn by the selected medical staff member, and make a call. The terminal device 50 of the medical staff member is a mobile terminal such as a PHS, a wearable terminal, or the like.

The medical staff member as the call target is not necessarily one person, and a plurality of persons can be selected. Accordingly, an administrative staff member can make a call (a group call) simultaneously to a plurality of medical staff members in the operating room OR-2. In a case where the call button 315A is pressed while any medical staff member is not selected, or where the call button 315A is pressed after a region other than the medical staff in the camera images is selected, a call to all the medical staff members in the operating room OR-2 is made.

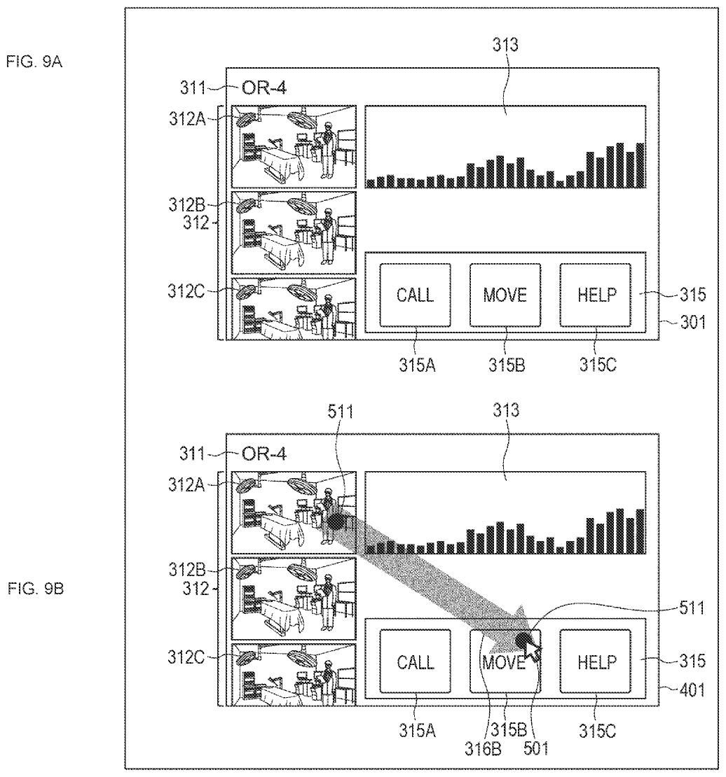

Alternatively, a hospital floor plan is displayed to allow selection of an operating room other than the operating room OR-2. Thus, a call can be made to the medical staff in each operating room of a plurality of operating rooms. Note that a medical staff member as the call target may be selected by a drag-and-drop action similar to an action to select a medical staff member as the relocation target as described later with reference to FIG. 9.

The move button 315B is a button to be handled when a medical staff member as the relocation target is moved (relocated) from the operating room which the medical staff member is currently in, to another operating room. FIG. 9 illustrates an example of a medical staff relocation action. At the time of performing a medical staff relocation action, image processing using camera images has been performed, the medical staff member displayed in the camera images has been identified, and the position of the medical staff member has also been identified.

In A of FIG. 9, a medical staff member is displayed in the camera image 312A disposed in the camera image group 312 on the area detail screen 301, and this medical staff member is to be moved to another operating room in this scene. As illustrated in B of FIG. 9, when an administrative staff member handles a mouse to move the pointer 501 to the medical staff member included in the camera image 312A, an action indicator 511 is displayed on the medical staff member, and the medical staff member is selected as the relocation target medical staff member.

The administrative staff member performs a drag-and-drop action on the action indicator 511, and move the action indicator 511 onto the move button 315B as indicated by an arrow in the drawing. Thus, the administrative staff member can move the medical staff member selected as the relocation target. For example, when the action indicator 511 is moved onto the move button 315B, a screen including the hospital floor plan is popped up so that the operating room as the relocation destination is selected. As a result, a voice instruction for requesting the selected medical staff member as the relocation target to move between the operating rooms is generated, and is reproduced by the terminal device 50 worn by the medical staff member as the relocation target. Thus, the medical staff member as the relocation target moves to the operating room as the relocation destination in accordance with the voice instruction.

Note that, among the medical staff members having roles, medical staff members estimated to be training staff members or visitors can be excluded from candidates for relocation. Also, medical staff members whose future task amounts are predicted to increase from a medical operation flow or the like can also be excluded from candidates for relocation. A visual effect may be added to an icon representing a medical staff member who is a candidate for relocation among the medical staff members.

The help button 315C is a button to be handled when assistance to the assistance target medical staff member is requested. For example, an administrative staff handles the pointer or the like, to select the assistance target medical staff member displayed in the camera images arranged in the camera image group 312. At this point of time, a voice instruction for requesting assistance from a medical staff member (a related medical staff member) having a role related to the assistance target medical staff member is generated, and is reproduced by the terminal device 50 worn by the related medical staff member. Thus, the related medical staff member will assist the assistance target medical staff member in accordance with the voice instruction. Note that the related medical staff member may be selected in a separate window or the like.

Note that a cancel button to be handled when an action is canceled may be disposed in the instruction button group 315. Also, in a case where there is a plurality of target medical staff members, and it is necessary to make allocation determination for the plurality of medical staff members, a separate window may be displayed so that the target medical staff members can be selected. Note that, in the region of the instruction button group 315, a medical operation flow, information indicating which stage of the medical operation flow the current operation status is in, and the like may be disposed.

Although the area detail screen 301 in a case where the area corresponding to the operating room OR-2 on the operation floor screen 201 in FIG. 4 is selected has been described herein, the area detail screen 301 corresponding to the selected area is also displayed in a case where some other area is selected.

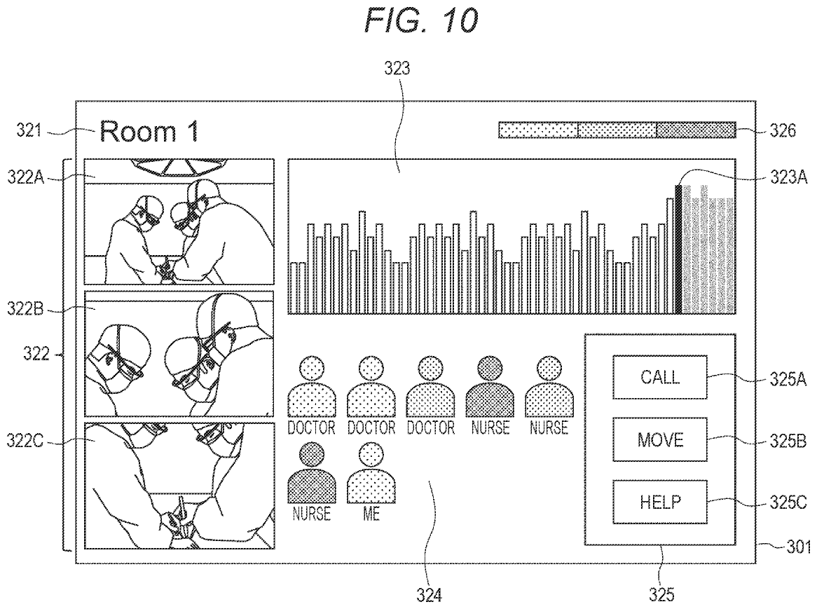

The display form of the area detail screen 301 illustrated in FIG. 8 is an example, and some other display form may be adopted. FIG. 10 illustrates another example of display of the area detail screen 301.

In FIG. 10, the area detail screen 301 displays an area name 321, a camera image group 322, an activity status graph 323, medical staff icons 324, an instruction button group 325, and a status gauge 326.

In the area name 321, “Room 1”, which is the name of the area corresponding to the operating room #1 is displayed. Camera images 322A to 322C captured in the operating room #1 are arranged in the camera image group 322. The activity status graph 323 is a graph indicating a temporal change in the amount of activity in the operating room #1. A bar 323A indicates the amount of activity at the current time, the left side thereof indicating the amount of the past activity, the right side thereof indicating the amount of the future activity (a predicted value).

The medical staff icons 324 show icons representing medical staff members such as doctors, nurses, and a medical engineer (ME) in the operating room #1. As there are seven medical staff members in the operating room #1 at the present moment, for example, seven human-shaped icons are displayed. The medical staff icons 324 are colored in accordance with the amounts of activity of the corresponding medical staff members. For example, the human-shaped icon corresponding to a medical staff member in a busy state is in red (a reddish color), and the human-shaped icon corresponding to a medical staff member in an unbusy state is in green (a greenish color).

In the instruction button group 325, a button group to be handled when an administrative staff member gives an instruction to a medical staff member in the operating room #1 is disposed. The instruction button group 325 includes a call button 325A, a move button 325B, and a help button 325C. Since the instruction button group 325 is similar to the instruction button group 315 illustrated in FIG. 8, explanation thereof is not made herein.

The status gauge 326 indicates the operation progress status in three stages. For example, the area detail screen 301 in FIG. 10 shows that the status gauge 326 has risen to the highest of the three stages, and the medical operation in the operating room #1 is close to the end. Note that the status gauge 326 may indicate a status other than the operation progress status, such as the status of the amount of activity in the operating room #1 in three stages.

Note that, in the activity status graph 323, a vertical bar indicating the current time may also be displayed. In a case where an administrative staff member performs an action to move the vertical bar representing the current time to the right side, the colors of the medical staff icons 324 may be changed on the basis of the amounts of the future activity of the respective medical staff members in accordance with the time indicated by the vertical bar.

As described above, on the area detail screen 301, the camera image group 312 (322) including one or a plurality of camera images of the target operating room, the activity status graph 313 (323) indicating a temporal change in the amount of activity in the operating room, the medical staff icons 314 (324) indicating the respective medical staff members in the operating room, and the instruction button group 315 (325) including action elements (buttons or the like) to be operated when an instruction is given to the medical staff in the operating room are arranged. Also, in the area detail screen 301, the background of the screen is set to a display form (a color or the like) depending on the amount of activity in the target operating room.

The medical staff icons 324 visually change with the amounts of activity of the respective medical staff members. Further, the medical staff icons 314 are in different display forms (colors or the like) in accordance with the roles of the respective medical staff members.

### (Staff Detail Screen)

FIG. 11 is a diagram illustrating an example of display of the staff detail screen 401.

In FIG. 11, the staff detail screen 401 includes graphics indicating the amount of activity of the target medical staff member.

The staff detail screen 401 in FIG. 11 is a screen to be displayed in a case where the icon representing a desired medical staff member is selected from among the medical staff icons 314 on the area detail screen 301 in FIG. 8, and a click action is performed. Alternatively, in a case where a predetermined action such as selecting a desired medical staff displayed in one of the camera images 312A to 312C on the area detail screen 301 in FIG. 8 is performed, the staff detail screen 401 illustrated in FIG. 11 may be displayed.

On the staff detail screen 401 in FIG. 11, the background is displayed in a background color corresponding to the situation of the target medical staff member. For example, in a case where it is determined, from the amount of activity of the target medical staff member, that the situation is a slightly busy state, the background color of the staff detail screen 401 is yellow (a yellowish color).

The staff detail screen 401 includes a medical staff member name 411, a camera image group 412, an activity status graph 413, and an instruction button group 414.

In the medical staff member name 411, “nurse” and “XXXXX”, which are the role and the name of the target medical staff member, are displayed. In the camera image group 412, camera images 412A to 412C captured in the operating room OR-2 in which the target medical staff member is located are disposed. The camera image 412A is a camera image showing the area around the hands during a medical operation, but may be a camera image captured by the terminal device 50 possessed by the target medical staff member.

The activity status graph 413 is a graph indicating the amount of activity of the target medical staff member in chronological order, the abscissa axis indicating time, the ordinate axis indicating the amount of activity. In the activity status graph 413, with the reference amount of activity being the amount of the current activity indicated by a vertical bar 413A, the left side indicates the amount of the past activity, and the right side indicates the amount of the future activity.

In a case where the pointer 501 is operated by an administrative staff member, and a desired bar 413B in the activity status graph 413 is selected, detailed information 413C regarding the target medical staff member indicated by the bar 413B is displayed. The detailed information 413C includes numerical information indicating the situation of the target medical staff member, and the like.

In the instruction button group 414, a button group to be handled when the administrative staff gives an instruction to the target medical staff member is disposed. The instruction button group 414 includes a call button 414A, a move button 414B, and a help button 414C. Since the instruction button group 414 is similar to the instruction button group 315 illustrated in FIG. 8, explanation thereof is not made herein. Note that, in the region of the instruction button group 414, a medical operation flow, information indicating which stage of the medical operation flow the current operation status is in, and the like may be disposed.

The display form of the staff detail screen 401 illustrated in FIG. 11 is an example, and some other display form may be adopted. FIG. 12 illustrates another example of display of the staff detail screen 401.

In FIG. 12, the staff detail screen 401 displays a medical staff member name 421, a camera image group 422, an activity status graph 423, an instruction button group 424, a status gauge 425, and a hand-region moving image 426.

In the medical staff member name 421, “doctor” and “XXXXX”, which are the role and the name of the target medical staff member, are displayed. In the camera image group 422, camera images 422A to 422C are disposed. In the activity status graph 423, a graph indicating the amount of activity of the target medical staff member in chronological order is displayed. A bar 423A indicates the amount of activity at the current time. In the instruction button group 424, a call button 424A, a move button 424B, and a help button 424C are disposed.

The status gauge 425 indicates the operation progress status in three stages. For example, the staff detail screen 401 in FIG. 12 shows that the status gauge 425 has risen to the highest of the three stages, and the medical operation is close to the end. Note that the status gauge 425 may indicate a status other than the operation progress status, such as the status of the amount of activity in the target medical staff member in three stages.

The hand-region moving image 426 is a camera image obtained by capturing the region around the hands during the medical operation. The hand-region moving image 426 may be a camera image captured by the terminal device 50 possessed by the target medical staff member.

Note that, with reference to the staff detail screen 401 in FIGS. 11 and 12, a case where one target medical staff member is selected, and the staff detail screen 401 related to the medical staff member is displayed has been described. However, the number of medical staff members to be selected is not necessarily one, and a plurality of medical staff members can be selected. In a case where a plurality of medical staff members is selected, graphics indicating the amounts of activity of these medical staff members may be displayed simultaneously, or may be displayed selectively.

As described above, on the staff detail screen 401, the camera image group 412 (422) including one or a plurality of camera images of the operating room in which the target medical staff is located, the activity status graph 413 (423) indicating a temporal change in the amount of activity of the medical staff member, and the instruction button group 414 (424) including action elements (buttons or the like) to be handled when an instruction is given to the medical staff are arranged. Also, in the staff detail screen 401, the background of the screen is set to a display form (a color or the like) depending on the amount of activity in the target medical staff member.

### (One-Screen Display)

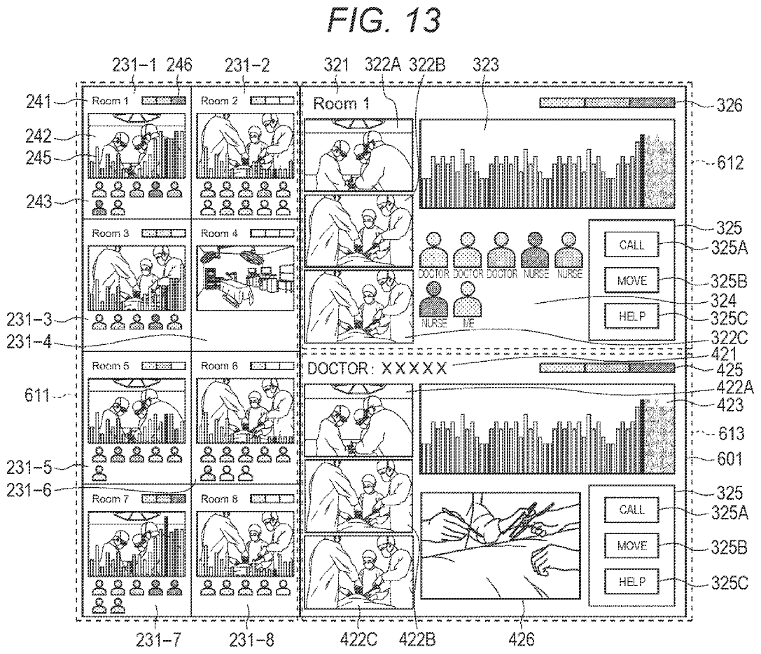

FIG. 13 is a diagram illustrating an example of one-screen display of information.

In FIG. 13, a screen 601 includes a first region 611 on the left side, a second region 612 on the upper right side, and a third region 613 on the lower right side.

The first region 611 corresponds to the area detail screen 301 in FIG. 7, and includes graphics indicating a temporal change in the amount of activity in the entire operating rooms and graphics indicating each of the medical staff members. However, the first region 611 differs from the layout of the area detail screen 301 in FIG. 7 in that the layout of the area information 231 is aligned with a vertically long region.

The second region 612 corresponds to the area detail screen 301 in FIG. 10, and includes graphics indicating a temporal change in the amount of activity in the target operating room, and graphics indicating the amount of activity of each medical staff member in the operating room. The third region 613 corresponds to the staff detail screen 401 in FIG. 12, and includes graphics indicating the amount of activity of the target medical staff member.

In this manner, not only the screen display is switched by selecting the area corresponding to an operating room or an individual medical staff member, but also the information about the respective screens can be collectively displayed on one screen. By checking the information displayed on the one screen, the administrative staff can determine the busyness in the operating rooms and the medical staff, using a larger amount of information as the determination material.

### (Icon Display)

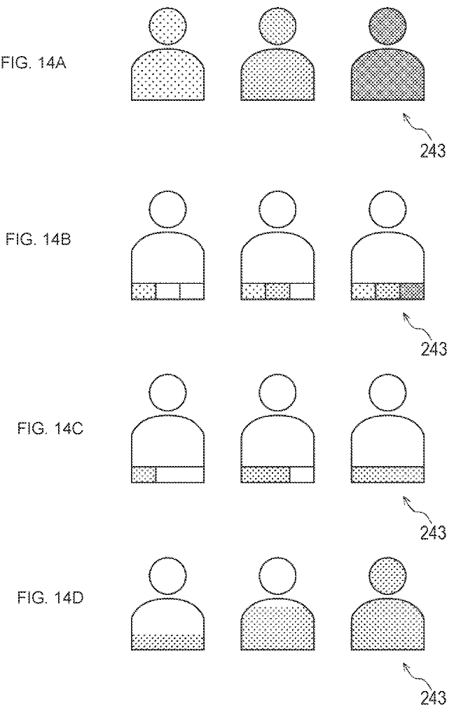

FIG. 14 is a diagram illustrating an example of display of medical staff icons.

Colors, patterns, or the like corresponding to the amounts of activity of the medical staff members are given to the corresponding medical staff icons 243. For example, the medical staff icons 243 can be shown in display forms as illustrated in A to D of FIG. 14. In A to D of FIG. 14, the amount of activity of a medical staff member is larger at a position closer to the right side in the drawing. Note that, in A to D of FIG. 14, the amount of activity of each medical staff member is indicated in three stages, but may be indicated in two stages or in four or more stages.

In A of FIG. 14, the medical staff icons 243 indicate the amounts of activity of the medical staff members by giving colors corresponding to the amounts of activity of the medical staff members to the entire regions of the human-shaped icons (shaped like human upper bodies).

In B of FIG. 14, the medical staff icons 243 indicate the amount of activity of the medical staff members by giving different colors corresponding to the amounts of activity of the medical staff members to the status bars in three stages shown in the regions on the lower side of the human-shaped icons. As illustrated in C of FIG. 14, each status bar in three stages may be integrally shown in the same color.

In D of FIG. 14, the medical staff icons 243 indicate the amounts of activity of the medical staff members by giving a predetermined color to the regions corresponding to the amounts of activity of the medical staff members in the entire regions of the human-shaped icons. Specifically, the medical staff icon 243 on the left side is colored up to the abdomen of the human-shaped icon, the medical staff icon 243 in the middle is colored up to the chest of the human-shaped icon, and the medical staff icon 243 on the right side is colored up to the head of the human-shaped icon. In this manner, the amount of activity of each medical staff member is indicated in three stages.

Note that the display forms of the medical staff icons 243 illustrated in FIG. 14 are examples, and any other display form that can visualize the amounts of activity of medical staff members may be adopted. Further, not only the medical staff icons 243 illustrated in FIGS. 6 and 7 but also the medical staff icons 324 illustrated in FIG. 10 can be presented in a similar manner.

### (Flow in a Process)

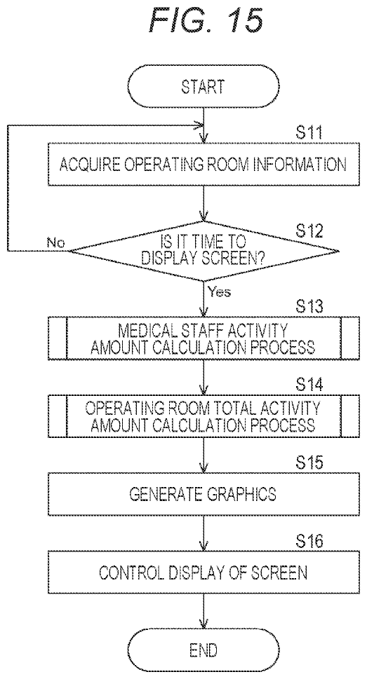

Referring now to a flowchart in FIG. 15, a flow in a process to be performed by the medical information processing system 1 illustrated in FIG. 1 is described.

In step S11, the operating room information acquisition unit 101 acquires operating room information. The operating room information includes camera image data.

In step S12, a check is made to determine whether it is time to display a screen. If it is determined in the determination process in step S12 that it is not the time to display a screen, the process returns to step S11. As a result, operating room information regarding each operating room is acquired at regular time intervals.

If it is determined in the determination process in step S12 that it is the time to display a screen, the process moves on to step S13.

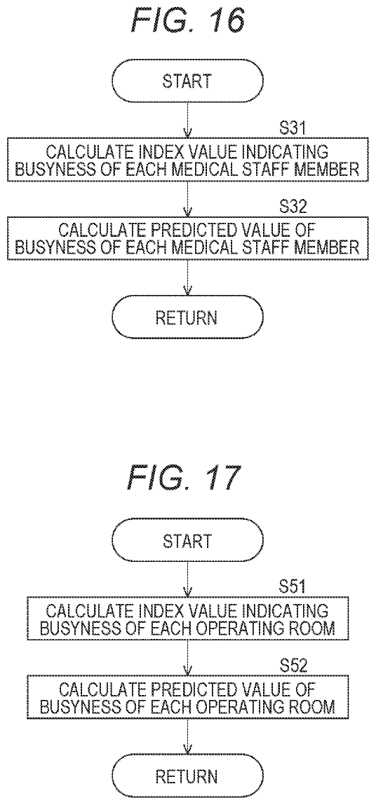

In step S13, the activity amount calculation unit 102 performs a medical staff activity amount calculation process, on the basis of the operating room information. In the medical staff activity amount calculation process, the amounts of activity of the medical staff members in the operating room are calculated. The medical staff activity amount calculation process will be described later in detail, with reference to a flowchart shown in FIG. 16.

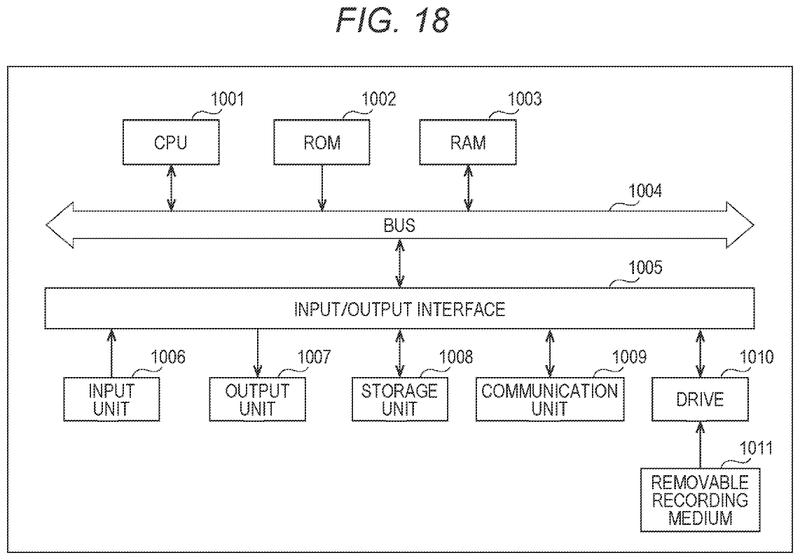

In step S14, the activity amount calculation unit 102 performs an operating room total activity amount calculation process, on the basis of the operating room information. In the operating room total activity amount calculation process, the amount of activity in the entire operating rooms in the hospital is calculated. The operating room total activity amount calculation process will be described later in detail, with reference to a flowchart shown in FIG. 17.

In step S15, the graphics generation unit 103 generates graphics. The graphics generation unit 103 generates medical staff graphics, on the basis of the amounts of activity of the medical staff members calculated in the medical staff activity amount calculation process. The graphics generation unit 103 generates operating room graphics, on the basis of the amount of activity in the entire operating rooms calculated in the operating room total activity amount calculation process.

In step S16, the screen display control unit 104 controls display of the screens to be displayed on the display device 30, on the basis of the generated graphics. The screens to be displayed on the display device 30 include the operation floor screen 201, the area detail screen 301, and the staff detail screen 401.

Here, the medical staff activity amount calculation process corresponding to step S13 in FIG. 15 is described in detail, with reference to a flowchart shown in FIG. 16.

In step S31, the activity amount calculation unit 102 calculates an index value indicating the busyness of each medical staff member, on the basis of the operating room information. With the index values, the amounts of the past and current activity of the respective medical staff members are obtained.

For example, the amount of activity of each medical staff member can be calculated from the amount of movement obtained by performing at least one of a person/object tracking process or an image analysis process using camera image data.

Specifically, the total sum of the sizes of motion vectors is calculated from the acceleration data included in the operating room information, and thus, the amount of movement of the target medical staff member or the like is obtained. In this manner, the amount of activity of the target medical staff member can be calculated. Alternatively, the target medical staff or the like as an object included in an image frame is identified from the camera image data included in the operating room information, and the total sum of the sizes of the motion vectors indicating how much the object has moved from the position of the object in the previous image frame is calculated. Thus, the amount of activity of the target medical staff member can be calculated.

That is, in a case where the total sum of the sizes of the motion vectors is larger than a threshold, the target medical staff member is performing an urgent task, and the amount of activity of the target medical staff member is large. On the other hand, in a case where the total sum of the sizes of the motion vectors is smaller than the threshold, the target medical staff member is not performing a particularly urgent task, and the amount of activity of the target medical staff member is small.

When the amount of activity of the target medical staff member is calculated, weighting may be performed on the basis of information regarding the task, such as the number of steps involved in the task and the contents of the task. For example, at least one of a process of tracking movement of the hands of the medical staff member, or an image analysis process using the camera image data is performed, so that the number of steps involved in the task and the contents of the task can be recognized.

For example, when the number of steps involved in the task is larger, there is a higher possibility that the task is very difficult or is being performed in a hurry. Accordingly, differences can be made by weighting. Further, depending on the contents of tasks, there are urgent tasks and non-urgent tasks. Therefore, weighting is performed on those tasks, to make differences between them. Examples of urgent tasks include a task with a wide range of required time and a task requiring cooperation of other medical staff members.

Note that the amount of activity of each medical staff member may be calculated, with some information taken into account, such as a recorded task history like a nursing record and an anesthesia record, and the situation of the medical operation flow (the occurrence of a task deviating from a standard medical operation flow, or the like).

In step S32, the activity amount calculation unit 102 calculates a predicted value indicating the busyness of each medical staff member, on the basis of the operating room information. With the predicted values, the amounts of the future activity of the respective medical staff members are obtained.

For example, the camera image data included in the operating room information is analyzed, so that the amount of the future activity of the target medical staff member can be predicted. When the camera image data is analyzed, it is possible to use information such as the medical operation flow, the condition of the patient (such as the amount of bleeding), and the state of the device (such as whether the device is appropriately functioning). Also, the amount of the future activity may be predicted from information such as the state of the medical operation flow and a change in the amount of activity in a similar medical operation in the past.

As the medical staff activity amount calculation process is performed in this manner, the amounts of the past, present, and future activity are calculated for each medical staff member in the operating room, and the amounts of activity of the respective medical staff member are obtained.

Next, the operating room total activity amount calculation process corresponding to step S14 in FIG. 15 is described in detail, with reference to a flowchart shown in FIG. 17.

In step S51, the activity amount calculation unit 102 calculates an index value indicating the busyness in each operating room, on the basis of the operating room information. With the index values, the amounts of the past and current activity in the respective operating rooms are obtained.

For example, the amount of activity in each operating room can be calculated from the amount of movement obtained by performing at least one of a person/object tracking process or an image analysis process using camera image data.

Specifically, the total sum of the sizes of motion vectors is calculated from the acceleration data included in the operating room information, and thus, the amount of movement of the medical staff members or the like in each operating room is obtained. In this manner, the amount of activity in each operating room can be calculated. Alternatively, the medical staff members and the like in the operating room are identified as objects included in an image frame from the camera image data included in the operating room information, and the total sum of the sizes of the motion vectors indicating how much the objects have moved from the positions of the objects in the previous image frame is calculated. Thus, the amount of activity in each operating room can be calculated.

That is, in a case where the total sum of the sizes of the motion vectors is larger than a threshold, urgent tasks are being performed by a large number of medical staff members, and the amount of activity in each operating room is large. On the other hand, in a case where the total sum of the sizes of the motion vectors is smaller than the threshold, any specific urgent task is not being performed by a large number of medical staff members, and the amount of activity in each operating room is small.

When the amount of activity in each operating room is calculated, weighting may be performed on the basis of information regarding the task, such as the number of steps involved in the task and the contents of the task. For example, at least one of a process of tracking movement of the hands of the medical staff member, or an image analysis process using the camera image data is performed, so that the number of steps involved in the task and the contents of the task can be recognized. Alternatively, when the amount of activity in each operating room is calculated, it is possible to use information such as the medical operation flow, the condition of the patient (such as the amount of bleeding), and the state of the device (such as whether the device is appropriately functioning).

For example, when the number of steps involved in the task is larger, there is a higher possibility that a large number of medical staff members are performing difficult tasks, or a large number of medical staff members are performing urgent tasks. Accordingly, weighting can be performed to make differences. Further, depending on the contents of tasks, there are urgent tasks and non-urgent tasks. Therefore, weighting is performed on those tasks, to make differences between them. Examples of urgent tasks include a task with a wide range of required time and a task requiring cooperation of other medical staff members.

Note that, when the amount of activity in each operating room is calculated, it is possible to use the values obtained when the amount of activity of each medical staff member was calculated. Also, the amount of activity in each operating room may be calculated, with some information taken into account, such as a recorded task history like a nursing record and an anesthesia record, and the situation of the medical operation flow (the occurrence of a task deviating from a standard medical operation flow, or the like).

In step S52, the activity amount calculation unit 102 calculates a predicted value indicating the busyness in each operating room, on the basis of the operating room information. With the predicted values, the amounts of the future activity of the respective operating rooms are obtained.

For example, the camera image data included in the operating room information is analyzed, so that the amount of the future activity in each operating room can be predicted. When the camera image data is analyzed, it is possible to use information such as the medical operation flow, the condition of the patient (such as the amount of bleeding), and the state of the device (such as whether the device is appropriately functioning). Further, subsequent medical operation flows may be estimated by analysis of the camera image data, tasks are assigned to the medical staff members in each estimated medical operation flow, and the amounts of activity may be calculated on the basis of the tasks and the amounts of movement. Also, the amount of the future activity may be predicted from information such as the state of the medical operation flow and a change in the amount of activity in a similar medical operation in the past.

As the operating room total activity amount calculation process is performed in this manner, the amounts of the past, present, and future activity are calculated for each operating room, and the amount of activity in the entire operating rooms is obtained.

As described above, in the medical information processing system 1, the amount of activity in the entire operating rooms and the amount of activity of each medical staff member are calculated on the basis of operating room information, and operating room graphics and medical staff graphics are generated on the basis of the calculated amounts of activity. A screen including at least either the generated operating room graphics or the generated medical staff graphics is then displayed. This makes it possible to visualize the situations in the entire operating rooms and the situations of the respective medical staff members.

For example, there may be a case where one medical staff member is performing, in a hurry, a task not greatly affecting the progress of the medical operation, while the entire operating rooms are calm. There may also be a case where the amount of activity in the entire operating rooms is large, while the tasks of the individual medical staff member are small. In the medical information processing system 1, on the other hand, the situations of the entire operating rooms and the situations of the respective medical staff members are visualized. Thus, the administrative staff can accurately grasp the situations of the entire operating rooms and the situation of each medical staff member, and take appropriate measures. Meanwhile, the amount of work of an individual medical staff member is not necessarily proportional to the amount of activity. However, in a case where the amount of work in the entire operating rooms is large, and the tasks spread over a wide range, for example, the amount of activity in the entire operating rooms can be precisely indicated.

### 2. Modifications

On the operation floor screen 201, the area detail screen 301, and the staff detail screen 401, a task deviating from a standard medical operation flow may be displayed as an event. The layout of the operation floor screen 201, the area detail screen 301, and the staff detail screen 401 can be changed as appropriate, but a certain condition can be added when the layout is changed. For example, it is possible to make it a condition that the activity status graph is disposed near the most relevant camera image, or that the instruction button group is disposed near the camera image group.

The number of medical staff icons to be selected at a time of transition from the area detail screen 301 to the staff detail screen 401 is not necessarily one, and a plurality of medical staff icons may be selected. In a case where a plurality of medical staff icons is selected, the staff detail screen 401 can display information regarding the plurality of the selected medical staff members.

Note that, when the amount of the future activity of each medical staff member or in each operating room is predicted, a trained model may be generated beforehand from the past operating room information, a medical operation flow, and the like as training data, and the amount of the future activity may be calculated with the trained model.

### (Computer Configuration)

The series of processes described above can be performed by hardware, or can be executed by software. In a case where the series of processes is performed by software, a program forming the software is installed into a computer of each device. FIG. 18 is a block diagram illustrating an example configuration of the hardware of a computer that performs the series of processes described above in accordance with a program.

In the computer in FIG. 18, a central processing unit (CPU) 1001, a read only memory (ROM) 1002, and a random access memory (RAM) 1003 are connected to one another by a bus 1004. An input/output interface 1005 is further connected to the bus 1004. An input unit 1006, an output unit 1007, a storage unit 1008, a communication unit 1009, and a drive 1010 are connected to the input/output interface 1005.

The input unit 1006 is formed with a microphone, various sensors, and the like. The output unit 1007 is formed with a speaker, a display, and the like. The storage unit 1008 is formed with a nonvolatile memory, a hard disk, and the like. The communication unit 1009 is formed with a network interface and the like. The drive 1010 drives a removable medium 1011 such as a magnetic disk, an optical disc, a magneto-optical disk, or a semiconductor memory.

In the computer designed as described above, the CPU 1001 loads a program recorded in the ROM 1002 or the storage unit 1008 into the RAM 1003, and executes the program, so that the series of processes described above is performed.

The program to be executed by the computer (the CPU 1001) may be recorded in the removable medium 1011 as a package medium or the like to be provided, for example. Also, the program can be provided via a wired or wireless transmission medium such as a local area network, the Internet, or digital satellite broadcasting.

In the computer, the removable medium 1011 is mounted in the drive 1010, so that the program can be installed into the storage unit 1008 via the input/output interface 1005. Also, the program can be received by the communication unit 1009 via a wired or wireless transmission medium, and be installed into the storage unit 1008. Alternatively, the program can be installed into the ROM 1002 or the storage unit 1008 in advance.

Here, in the present specification, the processes to be performed by a computer in accordance with a program include processes to be performed in parallel or independently of one another (parallel processing or object-based processing, for example). Furthermore, the program may be executed by one computer (processor), or may be executed by a plurality of computers in a distributed manner.

Note that embodiments of the present disclosure are not limited to the embodiment described above, and various modifications may be made without departing from the scope of the present disclosure. Furthermore, the effects described herein are merely examples and are not limited to specific effects, and some other effects may be provided. In the present specification, a system refers to a logical assembly of a plurality of devices.

Furthermore, the present disclosure can have the following configurations.

(1)

A medical information processing system including:
a calculation unit that calculates an amount of activity in entire operating rooms and an amount of activity of each medical staff member, on the basis of operating room information including an operating room camera image; and
a generation unit that generates operating room graphics including graphics indicating a temporal change in the amount of activity in the entire operating rooms, and medical staff graphics including graphics indicating each medical staff member, the medical staff graphics visually changing with the amount of activity.
   (2)

The medical information processing system according to (1), in which,
in the operating room graphics, the graphics indicating a temporal change in the amount of activity in the entire operating rooms is superimposed on the operating room camera image.
   (3)

The medical information processing system according to (2), in which,
in the operating room graphics, a graph indicating a temporal change in the amount of activity in each operating room is superimposed on a camera image of each operating room.
   (4)

The medical information processing system according to (3), in which
the graph indicates amounts of past, present, and future activity in each operating room.
   (5)

The medical information processing system according to any one of (1) to (4), in which
the operating room graphics includes graphics indicating a medical operation progress status.
   (6)

The medical information processing system according to any one of (1) to (5), in which
the medical staff graphics includes medical staff icons representing the respective medical staff members, and
the medical staff icons have different display forms in accordance with roles of the respective medical staff members.
   (7)

The medical information processing system according to any one of (1) to (6), further including
a display control unit that controls display of a screen including at least one of the operating room graphics or the medical staff graphics.
   (8)

The medical information processing system according to (7), in which
the display control unit displays a first screen including graphics indicating a temporal change in the amount of activity in the entire operating rooms, a second screen including graphics indicating a temporal change in an amount of activity in a target operating room and graphics indicating an amount of activity of each medical staff member in the target operating room, or a third screen including graphics indicating an amount of activity of a target medical staff member.
   (9)

The medical information processing system according to (8), in which,
on the first screen,
a camera image of an operating room and medical staff icons representing the respective medical staff members in the operating room are provided for each operating room,
a graph indicating a temporal change in the amount of activity in each operating room is superimposed on a camera image of each corresponding operating room, and
a background of the camera image of each operating room has a display form depending on the amount of activity in each corresponding operating room.
   (10)

The medical information processing system according to (9), in which
the medical staff icons visually change with the amounts of activity of the respective medical staff members.
   (11)

The medical information processing system according to (9) or (10), in which
the display forms of the medical staff icons vary with roles of the respective medical staff members.
   (12)

The medical information processing system according to any one of (8) to (11), in which,
on the second screen,
one or a plurality of camera images of the target operating room, a graph indicating a temporal change in the amount of activity in the target operating room, medical staff icons representing the respective medical staff members in the target operating room, and an action element to be handled when an instruction is given to a medical staff member in the target operating room are provided, and
a screen background has a display form that depends on the amount of activity in the target operating room.
   (13)

The medical information processing system according to (12), in which
the medical staff icons visually change with the amounts of activity of the respective medical staff members.
   (14)

The medical information processing system according to (12) or (13), in which
the display forms of the medical staff icons vary with roles of the respective medical staff members.
   (15)

The medical information processing system according to any one of (8) to (14), in which,
on the third screen,
one or a plurality of camera images of an operating room in which a target medical staff member is present, a graph indicating a temporal change in an amount of activity of the target medical staff member, and an action element to be handled when an instruction is given to the target medical staff member are provided, and
a screen background has a display form that depends on the amount of activity of the target medical staff member.
   (16)

The medical information processing system according to any one of (8) to (15), in which
the display control unit displays the first screen, the second screen, or the third screen in a switching manner, or displays the first screen, the second screen, and the third screen in one screen.
   (17)

A medical information processing method implemented in a medical information processing system,
the medical information processing method including:
calculating an amount of activity in entire operating rooms and an amount of activity of each medical staff member, on the basis of operating room information including an operating room camera image; and
generating operating room graphics including graphics indicating a temporal change in the amount of activity in the entire operating rooms, and medical staff graphics including graphics indicating each medical staff member, the medical staff graphics visually changing with the amount of activity.
   (18)

A program for causing a computer to function as:
a calculation unit that calculates an amount of activity in entire operating rooms and an amount of activity of each medical staff member, on the basis of operating room information including an operating room camera image; and
a generation unit that generates operating room graphics including graphics indicating a temporal change in the amount of activity in the entire operating rooms, and medical staff graphics including graphics indicating each medical staff member, the medical staff graphics visually changing with the amount of activity.

### REFERENCE SIGNS LIST

1 Medical information processing system
10 Information processing device
21 Camera device
22 Sensor device
30 Display device
40 Storage device
50 Terminal device
101 Operating room information acquisition unit
102 Activity amount calculation unit
103 Graphics generation unit
104 Screen display control unit
1001 CPU

1. A medical information processing system comprising:
a calculation unit that calculates an amount of activity in entire operating rooms and an amount of activity of each medical staff member, on a basis of operating room information including an operating room camera image; and
a generation unit that generates operating room graphics including graphics indicating a temporal change in the amount of activity in the entire operating rooms, and medical staff graphics including graphics indicating each medical staff member, the medical staff graphics visually changing with the amount of activity.

2. The medical information processing system according to claim 1, wherein,
in the operating room graphics, the graphics indicating a temporal change in the amount of activity in the entire operating rooms is superimposed on the operating room camera image.

3. The medical information processing system according to claim 2, wherein,
in the operating room graphics, a graph indicating a temporal change in an amount of activity in each operating room is superimposed on a camera image of each corresponding operating room.

4. The medical information processing system according to claim 3, wherein
the graph indicates amounts of past, present, and future activity in each operating room.

5. The medical information processing system according to claim 1, wherein
the operating room graphics includes graphics indicating a medical operation progress status.

6. The medical information processing system according to claim 1, wherein
the medical staff graphics includes medical staff icons representing the respective medical staff members, and
the medical staff icons have different display forms in accordance with roles of the respective medical staff members.

7. The medical information processing system according to claim 1, further comprising
a display control unit that controls display of a screen including at least one of the operating room graphics or the medical staff graphics.

8. The medical information processing system according to claim 7, wherein
the display control unit displays a first screen including graphics indicating a temporal change in the amount of activity in the entire operating rooms, a second screen including graphics indicating a temporal change in an amount of activity in a target operating room and graphics indicating an amount of activity of each medical staff member in the target operating room, or a third screen including graphics indicating an amount of activity of a target medical staff member.

9. The medical information processing system according to claim 8, wherein,
on the first screen,
a camera image of an operating room and medical staff icons representing the respective medical staff members in the operating room are provided for each operating room,
a graph indicating a temporal change in the amount of activity in each operating room is superimposed on a camera image of each corresponding operating room, and
a background of the camera image of each operating room has a display form depending on the amount of activity in each corresponding operating room.

10. The medical information processing system according to claim 9, wherein
the medical staff icons visually change with the amounts of activity of the respective medical staff members.

11. The medical information processing system according to claim 9, wherein
the display forms of the medical staff icons vary with roles of the respective medical staff members.

12. The medical information processing system according to claim 8, wherein,
on the second screen,
one or a plurality of camera images of the target operating room, a graph indicating a temporal change in the amount of activity in the target operating room, medical staff icons representing the respective medical staff members in the target operating room, and an action element to be handled when an instruction is given to a medical staff member in the target operating room are provided, and
a screen background has a display form that depends on the amount of activity in the target operating room.

13. The medical information processing system according to claim 12, wherein
the medical staff icons visually change with the amounts of activity of the respective medical staff members.

14. The medical information processing system according to claim 12, wherein
the display forms of the medical staff icons vary with roles of the respective medical staff members.

15. The medical information processing system according to claim 8, wherein,
on the third screen,
one or a plurality of camera images of an operating room in which a target medical staff member is present, a graph indicating a temporal change in an amount of activity of the target medical staff member, and an action element to be handled when an instruction is given to the target medical staff member are provided, and
a screen background has a display form that depends on the amount of activity of the target medical staff member.

16. The medical information processing system according to claim 8, wherein
the display control unit displays the first screen, the second screen, or the third screen in a switching manner, or displays the first screen, the second screen, and the third screen in one screen.

17. A medical information processing method implemented in a medical information processing system,
the medical information processing method comprising:
calculating an amount of activity in entire operating rooms and an amount of activity of each medical staff member, on a basis of operating room information including an operating room camera image; and
generating operating room graphics including graphics indicating a temporal change in the amount of activity in the entire operating rooms, and medical staff graphics including graphics indicating each medical staff member, the medical staff graphics visually changing with the amount of activity.

18. A program for causing a computer to function as:
a calculation unit that calculates an amount of activity in entire operating rooms and an amount of activity of each medical staff member, on a basis of operating room information including an operating room camera image; and
a generation unit that generates operating room graphics including graphics indicating a temporal change in the amount of activity in the entire operating rooms, and medical staff graphics including graphics indicating each medical staff member, the medical staff graphics visually changing with the amount of activity.
